(19) United States
(12) Patent Application Publication
(10) Pub. No.: US 2005/0234480 A1
(43) Pub. Date: Oct. 20, 2005

(54) Thread processing device

(76) Inventors: Sang-Hak Nam, Gumi-si (KR); Bong-Sik Kwon, Seongnam-si (KR); Jung-Min Song, Gumi-si (KR)

Correspondence Address: DERGOSITS & NOAH LLP, FOUR EMBARCADERO CENTER, SUITE 1450, SAN FRANCISCO, CA, 94111, US

(21) Appl. No.: 10/958,931

(22) Filed: Oct. 5, 2004

(30) Foreign Application Priority Data
Apr. 30, 2004 (KR) 10-2004-0030695
Apr. 16, 2004 (KR) 20-2004-0010589

(51) Int. Cl. A61B 17/04

(52) U.S. Cl. 606/148

(57) ABSTRACT

The present invention provides a thread processing device to provide thread with various shape(direction) and size of cogs to be used in facelift operation. The device comprises a metallic processing plate with one or more grooves formed in length direction. It is preferred for the processing plate to have graduations near the groove, have a planar or arc shape, and be resistant to corrosion. In another embodiment, the processing device also includes fixing stands, which meet with the both ends of the processing plate, and a supporting plate, which is perpendicular to the fixing stands. Clamps can be used to secure the thread.

### FIELD OF THE INVENTION

The present invention relates to a thread processing device employing a metallic processing plate with grooves and graduations, a supporting plate and fixing stands.

### BACKGROUND OF THE INVENTION

A new type of facelift operation has gained popularity in recent days, and demand of suture-like threads with barbs (or cogs or ridges) used in such operation is increasing rapidly. As demand for the threads with barbs is increasing, improved performance is also being required for these threads.

The most common procedure using these threads is lifting cheek tissue, which tends to droop toward the mouth with time and deepen the cheek crease. These threads are also used for other parts of the face. Eyebrows can be raised toward their original position to give a more open-eyed, less tired look. Drooped nose tips can be lifted with these threads and necks and jowls around the jawline may also be firmed up.

Many of the risks of traditional facelifts are associated with general anesthesia. However, the new facelift operation using threads having barbs (or cogs) on the surface is performed with mild local anesthesia. Therefore, it is safe, efficient and less expensive than the traditional facelift. In addition, there is little use for a scalpel and minimum cutting of the skin or scalp in comparison with the traditional facelift operation.

In regard to numbness and weakness of facial tissues are prevented and the possibility of infection is also reduced, because the new facelift operation requires a very small entry site for these threads and it is believed that the depth level of the threads inserted into tissues is above the nerve branches.

The threads with barbs (or cogs) are typically made of polypropylene, a material which has been time-tested for decades as a suture material. The low-weight high-resistance plastic threads have a barbed-wire design to be able to “anchor” itself to the facial tissue as the surgeon lifts the sagging anatomical structures in the appropriate directions to obtain the desired new look. The threads with “cogs” or “barbs” or “ridges” are mechanically optimal for the big weight and structural movement of the tissues of the face they must support.

However, the threads with barbs (or cogs) supplied in market are very expensive due to processing costs, and the high price of the threads is one of the factors that increase the price of facelift operations. Moreover, since there are few types of threads with different shape, size and direction of the barbs (or cogs) the doctors in the field have difficulty in applying these threads their various types of the operations. Accordingly, there is a need for a thread processing device to flexibly make various shape (direction) and size of the barbs (or cogs) at low cost

### SUMMARY OF THE INVENTION

Accordingly, it is an object of the present invention to provide a thread processing device to place barbs (or cogs) of various shape (direction) and size. The device of the present invention includes a metallic processing plate having at least one groove formed in length direction and clamps for securing the thread. In one embodiment, the processing plate also has graduations provided near the one or more grooves. In another embodiment, the plate can be planar or arc shaped and resistant to corrosion. In still another embodiment the thread processing device further includes two fixing stands, which meet with the both ends of the processing plate, and a supporting plate, which is perpendicular to the fixing stands. In this embodiment, the clamps can be mounted on the fixing stands to secure the thread.

### BRIEF DESCRIPTION OF THE DRAWINGS

The above and other objects and features of the present invention will become apparent from the following description of the invention, when taken in conjunction with the accompanying drawings, which respectively show:

FIG. 1 is a schematic illustration showing one embodiment of a device according to the present invention.

FIG. 2 is a cross-sectional diagram of one embodiment of a processing plate of the device, where threads of different diameters are placed on the grooves of different depths and diameters.

FIG. 3 is a schematic diagram of assembling the processing plate of one embodiment with the fixing stand at the point of the joining groove.

FIG. 4 is a schematic diagram of an alternative embodiment of the invention composed of processing plate and clamps.

FIG. 5 is a schematic diagram of a thread with barbs (or cogs) processed with a device according to the present invention.

### DETAILED DESCRIPTION OF THE INVENTION

Embodiments of the invention will be described below in detail with reference to the drawings.

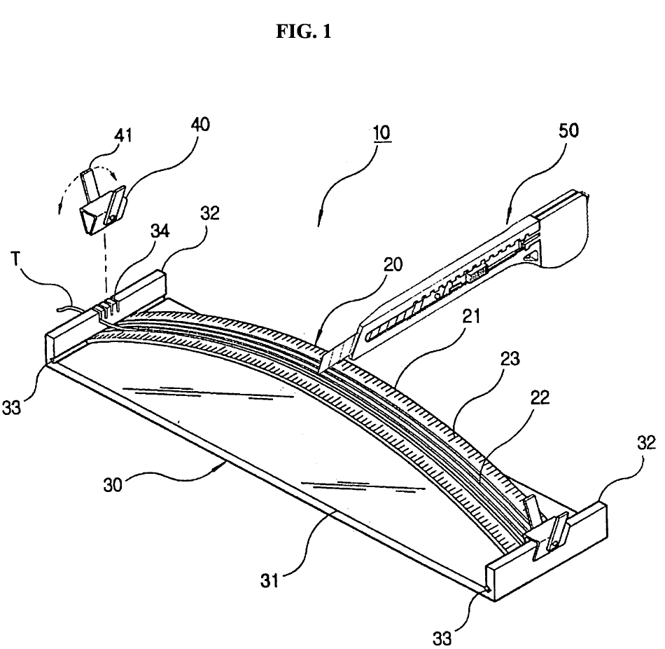

As shown in FIG. 1, a thread processing device (10), according to the embodiment, comprises a processing plate (21) made of thin metal plate resistant to corrosion with grooves (22) formed in length direction and graduations (23) thereon; two fixing stands (32) which meet with the both ends of the processing plate (21); supporting plate (31) which is perpendicular to the fixing stands (32); and clamps (40) having a rotating grip (41) which is to be mounted on the fixing stands (32). The processing plate (21) can be planar or arc shaped for the user's convenience. Furthermore, the processing plate can be mad eof any rigid material, but preferably a material that is resistant to corrosion. In addition, the device (10) can have a cutting element (50) for processing threads.

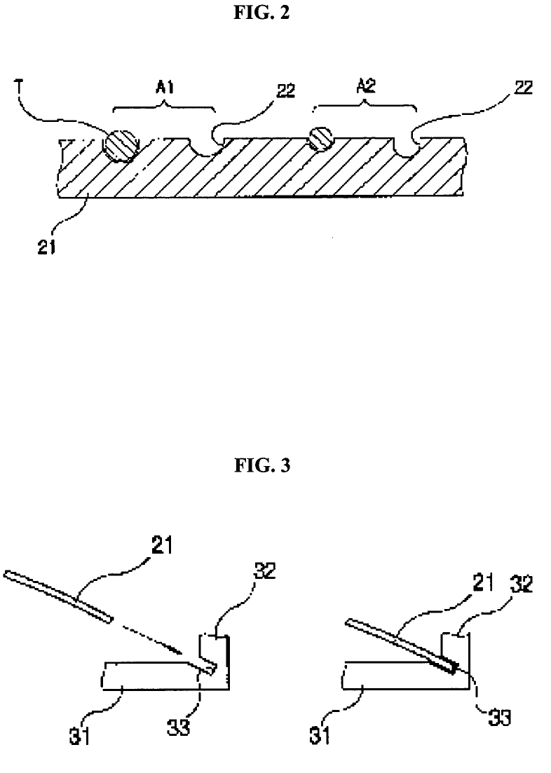

As shown in FIG. 2, two or more grooves (22) are formed on one side of the processing plate (21) and threads of various diameters can be placed on the grooves of the processing plate (21).

As shown in FIG. 3, the processing plate (21) can be inserted into joining groove (33) between the fixing stand (32) and supporting plate (31).

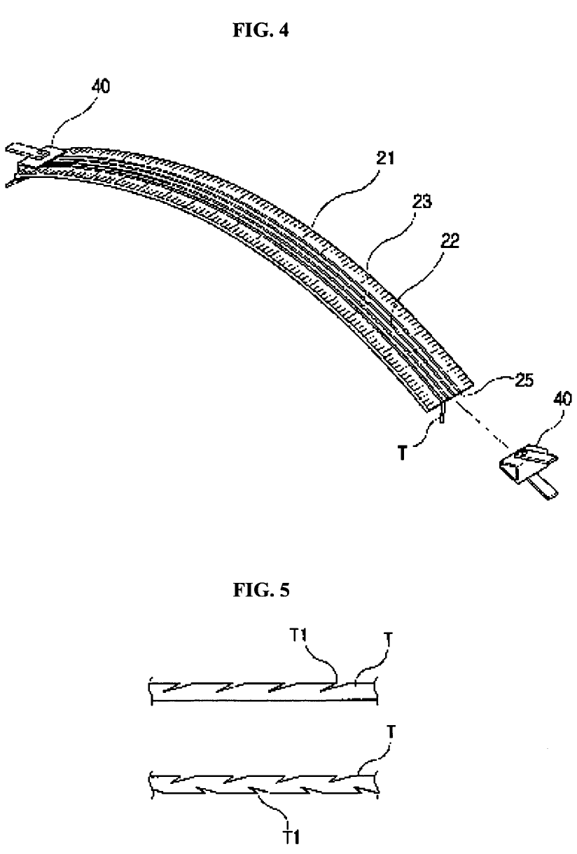

FIG. 4 illustrates an alternative embodiment of the invention, the processing plate (21) without supporting unit (30) can be independently used to cut the surface of the thread to desirable depth. In order to be used for such case, crevices (25) are formed at the both ends of the grooves (22), which are also at the both ends of the processing plate (21), for the purpose of fixing the thread, as shown in FIG. 4. The threads placed in the grooves (22) can be inserted into crevices (25) at the both ends of the processing plate (21), and can be fixed tightly by the clamps (40) with rotating grip (41), so the processing can be made without supporting unit (30).

As shown in FIG. 5, the threads processed by the device of the invention can have barbs (or cogs) of various shape (direction) and size. The threads can be processed to have barbs (or cogs) on one side or both sides (spiral type) of the surface in order to fit the user's need. It is believed that many microscopic ridges made along the full length of the thread at a certain angle can provide the smooth intradermal shift and grouping of soft tissues, to achieve better aesthetic shape and rigid fixation.

The supporting plate (31) and fixing stands (32) can be made of metallic material resistant to corrosion, and the plate and the stands may be made as a single body. In order to make as a single body, one metallic plate can be banded perpendicularly at the both ends to form fixing stands leaving the middle part to be supporting plate. The joining groove (33) is formed along the meeting point of the supporting plate (31) and fixing stand (32). Moreover, the fixing stand (32) has a number of fixing holes (34) correspondent to the number of grooves (22) of the processing plate (21) so the threads placed on the grooves (22) of the processing plate (32) can be also placed at the fixing holes (34) of each fixing stand (32), and then tightly fixed with the clamps (40). As shown in FIG. 3, both ends of the processing plate (21) could be inserted into the joining groove (33).

The clamp (40) used to fix threads can be made of thin metal material and is to be mounted on the fixing stands (32). The lower end of the clamp (40) is straight, so the end of the clamp (40) can reach the bottom of the fixing stand (32). The rotating grip (41) uses a hinge, and after the clamp (40) is mounted on the fixing stand (32), it is desirable that the grip (41) is pulled to one side to prevent any inconvenience in thread processing.

The thread can be placed on any one of the grooves (22) of the processing plate (21), the end part of the thread can be drawn out through the fixing hole (34) of the fixing stand (32), and clamps (40) can be mounted on the fixing stand (32) in order to fix the thread tightly during the processing.

The thread can be processed to have cogs in the surface by the cutting element (50) and the cutting procedure can be carried out by hand or an automated mechanical device. For example, an automated mechanical device can have one or more cutting elements and can also be programmed to provide the desired angle and depth of the cut at various intervals.

The device of the present invention is purposely designed so that the upper part of the thread placed on the grooves (22) of the processing plate (21) is protruded and the protruded part of the thread can be cut by the processing cutter to create barbs, cogs, or ridges (T1). Moreover, the cutting depth of the thread can be controlled by the different depths of the grooves (22) of the processing plate (21), and the space between cogs can be guided by the graduations (23) on the processing plate (21) according to the user's requirements.

The processing plate (21) can be used for thread processing independently without supporting plate (31) and fixing stands (32) as shown in FIG. 4. In case of using the processing plate (21) independently as illustrated in FIG. 4, the thread inserted in the groove (22) can be fixed by the crevice (25) and clamp (40).

Various kinds of threads with different shape (direction) and size of cogs can be produced using different depths and diameters of the grooves (22) of the processing plate (21).

While several embodiments of the present invention have been illustrated by way of example, it is apparent that further embodiments could be developed within the spirit and scope of the present invention. However, it is to be expressly understood that such modifications and adaptations are within the spirit and scope of the present invention, as set forth in the following claims.

1. A thread processing device comprising,
a processing plate with at least one groove formed in length direction fixing stands which meet the both ends of the processing plate;
supporting plate which is perpendicular to the fixing stands; and
clamps for securing the thread.

2. The device according to claim 1, wherein the processing plate has more than one groove and each grove has a different depth.

3. The device according to claim 1, wherein the processing plate has more than one groove and each groove has a different diameter.

4. The device according to claim 1, further comprising a cutting element for processing threads.

5. The device according to claim 1, wherein the processing plate further includes a plurality of graduations adjacent to the groove.

6. A thread processing device comprising,
a processing plate with at least one groove formed in length direction; and
clamps for securing the thread.

7. The device according to claim 6, wherein the processing plate has a crevice formed at the both ends of said grooves.

8. The device according to claim 6, wherein the processing plate has more than one groove and each grove has a different depth.

9. The device according to claim 6, wherein the processing plate has more than one groove and each groove has a different diameter.

10. The device according to claim 6, further comprising a cutting element for processing threads.

11. The device according to claim 6, wherein the processing plate further includes a plurality of graduations adjacent to the groove.
